(19) United States
(12) Patent Application Publication
(10) Pub. No.: US 2023/0052399 A1
(43) Pub. Date: Feb. 16, 2023

(54) METHODS TO ALTER THE TUMOR MICROENVIRONMENT FOR EFFECTIVE CANCER IMMUNOTHERAPY

(71) Applicant: PDS BIOTECHNOLOGY CORPORATION, Berkeley Heights, NJ (US)

(72) Inventors: Frank Bedu-Addo, Stamford, CT (US); Greg Conn, Madrid (ES); Siva K. Gandhapudi, Blue Ash, OH (US); Martin Ward, Lexington, KY (US); Jerold Woodward, Lexington, KY (US)

(21) Appl. No.: 17/894,828

(22) Filed: Aug. 24, 2022

Related U.S. Application Data
(63) Continuation of application No. 15/725,984, filed on Oct. 5, 2017, now abandoned.
(60) Provisional application No. 62/404,504, filed on Oct. 5, 2016.

(51) Int. Cl. A61K 39/395; A61K 39/00; A61K 39/39; A61P 35/00; A61K 45/06; A61K 39/12

(52) U.S. Cl. CPC A61K 39/39541; A61K 39/0012; A61K 39/39558; A61K 39/39; A61P 35/00; A61K 45/06; A61K 39/001; A61K 39/0008; A61K 39/12; A61K 2039/505; A61K 2039/585; A61K 2039/6018; A61K 2039/55572; C12N 2710/20034; A61K 2039/5154; A61K 2039/572; A61K 2039/55522; A61K 2039/55505

(57) ABSTRACT

Methods and compositions for altering the microenvironment of a tumor are provided. The methods comprise reducing the population of tumor-residing immune suppressive regulatory T-cells, increasing the population of tumor lysing T-cells (such as CD8+ T-cells) and improving the efficacy of cancer immunotherapy. The compositions comprise the use of cationic lipids optionally combined with autologous antigens, non-autologous antigens, or tumor-associated antigens.

### CROSS-REFERENCE TO RELATED APPLICATIONS

This application is a Continuation of U.S. patent application Ser. No. 15/725,984, filed Oct. 5, 2017, which claims benefit of priority under 35 U.S.C. § 119(e) of U.S. Ser. No. 62/404,504, filed Oct. 5, 2016, the entire contents of which is incorporated herein by reference in its entirety.

### INCORPORATION OF SEQUENCE LISTING

The material in the accompanying sequence listing is hereby incorporated by reference into this application. The accompanying sequence listing text file, named, G1421US00_PDS-17-1266R_CON.xml, was created Aug. 23, 2022, and is 1.66 kb in size. The file can be accessed using Microsoft Word on a computer that uses Windows OS.

### TECHNICAL FIELD

Embodiments of the present disclosure relate generally to novel methods for altering the miroenvironment of a tumor by reducing the population of tumor-residing immune suppressive regulatory T-cells. This disclosure also relates to novel methods for altering the miroenvironment of a tumor by reducing the population of tumor-residing immune suppressive regulatory T-cells while simultaneously increasing the population of tumor lysing such as CD8+ T-cells.

### BACKGROUND

Current scientific evidence supports the view that the human immune system produces a population of T cells, called regulatory T cells (Tregs), that are specialized for immune suppression. Disruption in the development or function of Tregs is a leading cause of autoimmune and inflammatory diseases in humans. The involvement of Tregs in tumor immunity was originally reported in 1999 by Shimizu et al. (J. Immunol. 163:5211). In addition, CD4(+) regulatory T cells (Tregs) that express the transcription factor FoxP3 are known to be highly immune suppressive and play an important role in the maintenance of self-tolerance. However, in malignant tumors they promote cancer by suppressing effective antitumor T-cell immunity. Mice treated with anti-CD25 antibody (which depleted the CD4⁺CD25⁺T_{regs}) and nude (T cell deficient) mice that were given splenocytes that had been treated with anti-CD25, exhibited tumor rejection and retardation of tumor growth, and interestingly the latter mice simultaneously exhibited autoimmunity in the stomach and the thyroid.

Higher infiltration by Tregs is also observed in tumor tissues, and in animal models, their depletion augments antitumor immune responses. Additionally, increased numbers of Tregs and decreased ratios of CD8(+) T cells to Tregs within the tumor has been correlated with poor prognosis in several types of human cancers. In cancer tissues, immune suppressive cytokines, molecules and cells including Tregs constitute the immunosuppressive network to inhibit effective antitumor immunity, thereby promoting cancer progression (Shimzu et al.). Tregs engaged in self-tolerance favorably control the activation of T cell responses to cancer antigens that are derived from self-constituents (so-called shared antigens), but may be less suppressive to T cells recognizing foreign antigens. (Maeda Yet al. 2014. Science 346:1536) What is needed is an integration of approaches reducing the suppressive activity and/or number of Tregs with approaches such as blocking immune checkpoint molecules, in order to broaden the therapeutic spectrum of cancer immunotherapy to cancer patients.

In a recent review of therapeutic cancer vaccines Melief et al, (Therapeutic Cancer Vaccines J Clin Invest. 2015; 125(9):3401-3412) stated the following; “Suboptimal vaccine design and an immunosuppressive cancer microenvironment are the root causes of the lack of cancer eradication. Drugs or physical treatments that can mitigate the immunosuppressive cancer microenvironment and include chemotherapeutics, radiation, indoleamine 2,3-dioxygenase (IDO) inhibitors, inhibitors of T cell checkpoints, agonists of selected TNF receptor family members, and inhibitors of undesirable cytokines. The specificity of therapeutic vaccination combined with such immunomodulation offers an attractive avenue for the development of future cancer therapies. Although such immunomodulation has been recognized as an “attractive avenue” no effective mechanism by which to traverse that avenue has yet been reduced to practice.

Because of the many T cell-suppressive activities in the cancer microenvironment, cancer vaccines cannot be expected to show optimal anticancer efficacy by themselves, but need to be used in combination treatments that are designed to inactivate the most important immunosuppressive mechanisms in this environment. Many standard chemotherapeutic agents such as thalidomide derivatives and other targeted compounds are known to deplete immunosuppressive Tregs and/or MDSCs without affecting effector T cell and memory T cell populations. However, there remains a need for novel methodologies for collectively and strategically attacking a tumor, methods that make the tumor's microenvironment hostile and less conducive for tumor proliferation, while simultaneously improving the efficacy of available cancer therapies.

Unlike prophylactic vaccines that are administered to healthy individuals, immunotherapies including cancer vaccines are administered to cancer patients to treat the disease. Therapeutic cancer vaccines are designed to induce cytolytic T-cell and memory responses. Cancer vaccines have demonstrated clear indications of clinical efficacy in the treatment of cancer, however, challenges remain. Various immune effector mechanisms critical to be induced by therapeutic vaccination or immunotherapy are required to specifically attack and destroy cancer cells without destroying normal healthy cells. The goal of therapeutic cancer immunotherapy, in principle, is to inhibit progression of advanced cancers and/or relapsed tumors that are refractory to conventional therapies, such as surgery, radiation therapy and chemotherapy, and to cure early and late stage cancer where possible.

Therapeutic vaccination has demonstrated excellent results in pre-cancer. However, therapeutic vaccination in the metastatic setting has not yet demonstrated clinical significance. This has been attributed to the heavy tumor burden which generates a tumor microenvironment which hosts various immune suppressor mechanisms that hamper anti-tumor cytolytic T-cell responses. This effect has been referred to as “immune escape” or “immune tolerance”. In an attempt to avoid the inhibitory effects existing in late stage tumors, clinical trials have been performed with patients using therapeutic vaccination as an adjuvant or add-on therapy in cases with minimal residual disease and a high risk of relapse. (Sears et al. Expert Opin Biol Ther. 2011; 11(11):1543-1550) The rationale behind vaccination in this clinical setting is that patients with minimal tumor burden still have a fully competent immune system capable of developing robust antitumor responses. Moreover, vaccinating in the adjuvant setting or early-stage cancer has the advantage of minimizing the accumulation of T cells within immune-suppressive tumor environments where they might be inactivated. Recent reports from clinical trials support the application of therapeutic vaccination as an adjuvant therapy in patients with low tumor load post-surgery or in patients with more indolent disease. (Sears et al. Expert Opin Biol Ther. 2011;11(11):1543-1550) In these settings, therapeutic vaccines have significantly reduced the frequency of recurrences. Importantly, booster inoculations are essential to maintain any immunity with peptide cancer vaccines. In the NeuVax Phase II trial with disease-free patients at high risk for recurrence, immunity was noted to wane with time and this corresponded with increased recurrences noted in the vaccine arm. Booster inoculations could maintain immunity, and those who received scheduled booster inoculations were less likely to recur. (Sears et al. Expert Opin Biol Ther. 2011;11(11):1543-1550) These findings are in line with the emerging body of evidence supporting immunotherapy in patients with a low tumor burden. Proving efficacy of cancer vaccines alone in this setting may allow the use of novel adjuvants and combination therapy to expand the indications to more aggressive and advanced diseases. Results from various clinical trials have suggested to most in the field that monotherapies are unlikely to confer significant clinical benefits to patients because of the serious obstacles provided by the tumors which diminish antitumor immunity. (Khalil et al. Adv Cancer Res.2015;128:1-68) Moreover, tumor cells generate adaptive immune resistance, a process which enables them to evade immune attacks. (Ribas A. et al. Cancer Discov. 2015;5(9):915-919) Therefore, interventions are needed to re-instate anticancer immune responses by actively counteracting the immune inhibitory mechanisms of tumor cells. In this respect, clinically effective antitumor responses are dependent on the modulation of more than one immune pathway which will enable the induction of robust T-cell responses against the tumor. There exists a need therefore for methods to improve the clinical efficacy of cancer vaccines by combine the use of such vaccines with other modalities, especially those that combat tumor immune suppression. Despite the failures from vaccination studies, it is recognized that cancer vaccines may generate meaningful antitumor responses under the appropriate conditions in the context of patients who have a functioning immune system that can respond to the vaccine. In addition, there is a need for combination regimens with agents that minimize a tumor's immuno-suppressive capabilities such as immune checkpoint inhibitors (Jochems C et al. Cancer Immunol Immunother.2014;63(4):407-418) so that vaccines with having suboptimal immunological responses, may be further enhanced.

Besides immunomodulatory antibodies, several other immune-modulating molecules targeting oncogenic pathways have been approved for treatment. (Khalil , et al.2015; 128:1-68) Several therapies that target inhibitory pathways such as Tregs and MDSCs are being developed. The combined use of these medicines with cancer vaccines is the subject of broad investigation and holds promise for the improvement of cancer vaccines.

Also needed are methods and compositions to effectively present tumor antigens to antigen presenting cells (APC) to enable more effective presentation via MHC class I (CD8+ T-cells). Currently available methodologies are suboptimal, and directly influence the potency and robustness of the resulting T-cell response.

Cross presentation refers to a pathway in which soluble proteins or peptides enter the cell from the outside and enter the MHC class I processing pathway. This can occur two ways, via the cytosolic pathway or the endosomal pathway. In both pathways, the proteins are initially taken up in endosomes/phagosomes. In the cytosolic pathway, a portion of partially degraded endosomal proteins ultimately enter the cytoplasm, via poorly understood mechanisms, where they are processed through proteasomes and the resulting peptides transported by TAP into either the endoplasmic reticulum or other endosomes for binding to MHC class I. Alternatively, proteins can be endosomally degraded and peptides can bind to MHC class I present in the endosomes. This latter pathway is proteasome independent and inefficient as it relies on the chance production of the correct peptide by endosomal proteases. Entry of proteins into early endosomes which contain limited proteolytic activity favor cross-presentation, while late endosomes which contain higher levels of proteolytic activity may inhibit cross-presentation.

From the above discussion, it is clear that proteins entering the endosomal pathway, particularly the early endosomal pathway, can be cross-presented on MHC class I. It also follows that the degree of cross presentation depends on the amount of a particular protein/peptide taken up into early endosomes, and the quantity of antigenic fragments subsequently delivered to the cytoplasm.

Soluble proteins which bind to dendritic cell (DC) scavenger receptors can also be cross-presented. The classic example is ovalbumin which binds to the mannose receptor. However not all proteins/peptides will bind to DC scavenger receptors which has led to various approaches of receptor targeting. These approaches include targeting the Fc receptor, various C-type lectin receptors like CD205, CD207, CLEC9a, integrins, or glycolipids. There are several drawbacks to these approaches. There is a requirement for coupling the antigen to a receptor binding protein, usually a monoclonal antibody, resulting in a potentially cumbersome approach. The amount of protein uptake is limited to the amount of receptor internalization that can occur, and once internalized, there is limited egress into the cytoplasm. Some DC receptors target late endosomes resulting in inefficient cross-presentation. Finally, the distribution of cross-presenting DC receptors on human DC subsets is poorly understood making the design of such technologies difficult, and can explain why mouse studies have not translated well to humans.

Another less specific approach is to convert the soluble antigen to a particulate form through attachment to nanoparticles. This approach suffers from the difficulty of delivering sufficient antigen and the fact that DC lose their phagocytic ability as they mature and traffic to lymph nodes.

What is needed are improved methods for antigen presentation to components of the immunological system, such as for example, dendritic cells.

### SUMMARY OF THE INVENTION

The ability to significantly alter the microenvironment of a tumor by reducing the population of tumor-residing immune suppressive regulatory T-cells while simultaneously increasing the population of tumor lysing T-cells (such as CD8+) is critical to effective cancer immunotherapy. The current disclosure provides novel methods comprising the use of cationic lipids to effectively alter the tumor microenvironment leading to effective cancer immunotherapy. In an embodiment, the cationic lipids utilized in the methods can effectively lower the population of regulatory T-cells (Treg) present within the tumor-microenvironment. In certain embodiments, where cationic lipids are combined with tumor-specific or tumor-associated antigens the cationic lipids can simultaneously facilitate the presentation of the tumor antigens to T-cells resulting in effective infiltration of tumor-targeting CD8+ and CD4+ T-cells. The novel methods described herein utilize the ability of cationic lipids of the disclosure to significantly decrease the ratio of immune-suppressive Treg to tumor-lysing T-cells leading to a more effective approach to anti-tumor immunotherapy.

Novel methods and compositions for lowering the population of Tregs within the tumor microenvironment are provided herein. The novel methods demonstrate that cationic lipids can be used as immunotherapeutic agents to safely reduce the population of immune-suppressive Tregs within tumors. In certain embodiments, cationic lipids as described herein are combined with tumor-specific (or tumor-associated) antigens facilitating simultaneous antigen uptake and processing by dendritic cells as well as presentation of such antigens to CD4+ and CD8+ T-cells in the context of MHC Class I and Class II; though not wishing to be bound by the following theory, such antigen presentation enables the induction of high levels of tumor-infiltrating T-cells. These effects significantly alter the tumor microenvironment by causing a low Treg to CD8+ T-cell ratio which facilitates a highly effective anti-tumor therapeutic response without the use of multiple, or combination therapies.

In an embodiment, the methods disclosed herein enable the development of immunotherapies which are capable of being used as a monotherapy to treat cancer by performing key immunological functions necessary to facilitate effective lysis of tumor cells by cytolytic T-cells.

Cationic lipids have been reported to facilitate antigen uptake and presentation via MHC class I and class II. Certain cationic lipids have also been reported to act as potent immunological adjuvants. However, the ability of cationic lipids to reduce the population of immune suppressive regulatory T-cells within the tumor's microenvironment was previously unknown.

Significantly, as demonstrated herein, some cationic lipids have been shown to have negligible ability to prime antigen-specific T-cells, and hence low ability to alter the tumor's microenvironment by lowering the Treg to CD8+ T-cell ratio. This function is therefore not an inherent property of cationic lipids, and the inventors herein have identified for the first time the use of cationic lipids that perform both effective antigen-specific T-cell induction as well as inhibition of Tregs as immunotherapeutic agents.

As would be evident to those skilled in the art, it is not possible to identify every cationic lipid that meets the described and required characteristics: the current disclosure enables and teaches a newly discovered application of cationic lipids and the methods utilized to identify suitable lipids. Those knowledgeable in the field will be able to perform the experiments necessary to identify suitable cationic lipids for altering the tumor's microenvironment based on the teachings provided herein.

Disclosed herein are improved methods for antigen presentation to components of the immunological system, such as for example, dendritic cells. As demonstrated herein, certain cationic lipids are unique in their ability to rapidly bind to dendritic cells in a receptor independent fashion and are rapidly taken up into early endosomes. Importantly, the inventors show that once in early endosomes, cationic lipids facilitate the destabilization of endosomes and delivery of contents into the cytoplasm for entry into the class I processing pathway. This allows for much more of the endosomal content to be delivered to the cytoplasm than would occur with targeted receptor uptake. The suitable cationic lipids are also able to provide the immunological signals that induce the production of certain cytokines and chemokines that provide activation and proliferation of T-cells and also cause the migration of T-cells into the lymph nodes.

The novel methods and compositions provided herein comprise suitable cationic lipids that are capable not only of facilitating antigen cross-presentation as described above, but also of simultaneously reducing the population of Treg cells within the tumor microenvironment. The disclosure allows for the critical functions of immunotherapy to be performed with a simple lipid based monotherapy—superior CD8+ T-cell induction and minimization of the tumor's immune suppressive microenvironment.

The inventors herein provide a novel discovery supported by validating data demonstrating that select cationic lipids, when combined with a tumor antigen to form a cancer vaccine are capable of effectively altering the tumor microenvironment by increasing the amount of tumor specific CD8+ T-cell within the tumor's microenvironment as well as a significant reduction in the Treg population, thus resulting in a significantly reduced Treg to CD8+ T-cell ratio. This provides effective anti-tumor response as a monotherapy.

The studies provided herein demonstrate that cationic lipids on their own may provide strong ability to lower the Treg population within the tumor. In addition to enabling the reduction of the Tregs, the present inventors demonstrate that when combined with an effective tumor targeting vaccine by adding tumor antigens to the cationic lipid, a highly effective anti-cancer therapy results by suppressing the Treg population within the tumors, while maximizing the CD8+ T-cell tumor-infiltrating population. This discovery provides significant benefit in the development of new cancer vaccines capable of inducing regression of advanced tumors.

### BRIEF DESCRIPTION OF THE DRAWINGS

FIG. 1 provides a graph showing that DOTAP enhancement of antigen processing is specific for dendritic cells. Bone marrow derived dendritic cells were incubated with DQ-OVA in the presence of various concentrations of DOTAP as shown. In addition, a mouse epithelial cell line, TC1, was treated in an identical fashion. Plot shows the green fluorescent intensity of gated CD11c cells (DC) or total TC1 cells.

FIG. 2 provides fluorescence readings for receptor mediated uptake. R-DOTAP nanoparticles, but not MPL stimulate uptake and processing by DC within 10 minutes. Mouse bone marrow DC were incubated in the presence of fluorescent DQ-OVA for one hour at either 37° C. or 4° C./azide in the presence of 25 uM DOTAP nanoparticles, 10 μg/ml MPL or media alone, and analyzed by flow cytometry. On the presented density plot, the y-axis represents the amount of green fluorescence which results from uptake and processing of the protein, while the x-axis represents the amount of red fluorescence resulting from concentration in endosomal vesicles.

FIG. 3: R-DOTAP enhancement of antigen processing results in enhancement of cross-presentation to MHC class I restricted T cells. Bone marrow derived DC were incubated in the presence of various concentrations of whole OVA protein and 25 uM DOTAP or DOTMA for 30 min at 37° C. or 4oC. DC were then washed and added to OT1 splenocytes (TCR transgenic T cells specific for the class I restricted OVA peptide SIINFEKL (SEQ ID NO: 1) in microtiter plates and cultured for three days at 37° C. The plot shows the mean CPM of ³H-thymidine uptake during the final 18 h of culture. Control cultures contained OT1 splenocytes and SIINFEKL (SEQ ID NO: 1) (10 uM) only, which bypasses the need for antigen processing. Both DOTAP and DOTMA enhanced the presentation of OVA by DC in a dose dependent manner, and this process is largely inhibited at 4° C., suggesting an active metabolic process is required.

FIG. 4 provides a graph showing that R-DOTAP induces superior stimulation of antigen-specific CD8+ T-cells. Effect of HPV16-E7, R-DOTAP/HPV16-E7, S-DOTAP/HPV16-E7 and Alum/MPL/HPV16-E7 vaccination on HPV16-specific CD8+ T-cell induction by ELISpot. The HPV16 E7 peptide used in the vaccine (SEQ ID NO: 2) is labelled KF18. Superior CD8+ T-cell induction is demonstrated with R-DOTAP.

FIG. 5: provides a graph showing that R-DOTAP/E7 induces superior regression of HPV-positive tumors compared to S-DOTAP/E7. Effect of R-DOTAP (solid squares), R-DOTAP/HPV16-E7 (clear squares) and S-DOTAP/HPV16-E7 (solid circles) vaccination on regression of established HPV16-positive TC-1 tumors. The HPV16 E7 peptide used in the vaccine (SEQ ID NO: 2) is labelled KF18. Tumor volumes were measured using calipers. Tumor regression is demonstrated with R-DOTAP/E7 and lack of regression or inhibition of growth observed with R-DOTAP (without antigen) and S-DOTAP/E7.

FIG. 6: provides a graph showing that R-DOTAP and R-DOTAP+antigen uniquely reduce the population of regulatory T cells within the tumors after vaccination. 1×10⁵ cells implanted subcutaneously on the right side of the abdomen of female B6 mice on day 0 and were vaccinated on day 12 and on day 19 post Tumor implant. T regulatory cells (CD45+ CD3+CD4+CD25+Foxp3+) cells infiltrated into tumors on day 19. The reduction is calculated as the ratio of total T reg cells in tumor/average T reg cell in tumor and normalized to T reg cells in naive mouse. Data represents mean±SEM of 4-5 mice in each group. *Data represents mean±SEM of 4-5 mice in each group. *Statistically significant R-DOTAP+antigen compared to all other groups (other than R-DOTAP only). P<0.01.

FIG. 7 provides a graph showing that R-DOTAP/E7 vaccination results in superior induction of tumor-infiltrating HPV16-specific CD8+ T cells in tumor-bearing mice. 10⁵ TC-1 cells were implanted subcutaneously on day 0. Mice were vaccinated on day 12 and on day 19. Antigen specific T cells infiltrating into the tumor were measured using RF9 specific dextramers and flow cytometry on Day 19. Data represents mean±SEM of 4-5 mice in each group.

FIG. 8 provides a graph showing that R-DOTAP/E7 vaccination results in superior reduction of the ratio of regulatory T-cells to HPV16-specific CD8+ T-cells within the tumor microenvironment. Ratio of T regulatory cells (Tregs) to HPV16 E7-specific CD8+ T cells among CD45+ cells that have infiltrated into the tumor microenvironment. Data represents mean±SEM of 4-5 mice in each group.

FIG. 9 provides a graph showing that R-DOTAP/E7 vaccination results in superior regression of established TC-1 tumors compared to GM-CSF/E7 or antigen alone. Tumors were implanted on Day 0 and the mice vaccinated on Days 12 and 19. Tumor volumes were measured using calipers. The naive mice group are tumor bearing mice that remained untreated. The HPV16 E7 peptide used in the vaccine (SEQ ID NO: 2) is labelled KF18. *Statistically significant R-DOTAP+antigen tumor regression compared to all other groups. P<0.01.

FIG. 10 provides a graph showing that R-DOTAP injection induces the migration of lymphocytes including T-lymphocytes into the draining lymph nodes. FIG. 10 shows the quantification of T-lymphocyte infiltration into the draining lymph node at 15 hours and total lymphocyte infiltration at various time points up to 4 days. R-DOTAP injection is represented by striped bars, and the control is represented by shaded bars.

### DETAILED DESCRIPTION

The following detailed description is exemplary and explanatory and is intended to provide further explanation of the present disclosure described herein. Other advantages, and novel features will be readily apparent to those skilled in the art from the following detailed description of the present disclosure. References mentioned herein, including U.S. Provisional Application Ser. No. 62/404,504 are incorporated by reference in their entirety.

Disclosed herein are novel methods and compositions comprising the use of cationic lipids, for altering and modifying the environment of a tumor in order to improve anti-cancer efficacy of therapeutics. In an embodiment, the tumor environment, or microenvironment is modified by reducing the population of Tregs within the tumor. In a further aspect, the methods and compositions disclosed herein comprise the use of cationic lipids to promote antigen cross-presentation and to increase the population of tumor-specific T-cells, including but not limited to CD8+ T-cells, within and around the tumors. In an embodiment, cationic lipids may be combined with tumor antigens to direct cytolytic activity against the specific tumor cells.

Cationic liposomes have been extensively used in-vivo for delivering small molecular weight drugs, plasmid DNA, oligonucleotides, proteins, and peptides and as vaccines. Recently cationic lipids have also been reported to be strong vaccine adjuvants. The inventors herein provide for the first time, studies demonstrating that certain cationic lipids facilitate the ability of promoting the proliferation of effector T-cell phenotypes in preference to immune suppressive Tregs and actively reduce the population of Tregs within the tumors thereby enabling cytolytic T-cell activity. To date, there has been no reported use or reports of the ability of cationic lipids as immunotherapies to directly alter the tumor microenvironment by reducing the population of Treg cells and/or promoting cytolytic T-cell activity.

Various embodiments of the invention are described herein as follows. In one embodiment a composition comprising one more cationic lipids is administered to reduce the population of Tregs within the tumor microenvironment.

In another embodiment, a composition comprising one more cationic lipids is combined with a tumor antigen to induce effective reduction of the Treg to CD8+ T-cell ratio within the tumor microenvironment.

In another embodiment, a method of treating cancer, the method comprises the step of treating the subject with a cationic lipid combined with a protein antigen.

In another embodiment, a method of treating cancer, the method comprises the step of treating the subject with a cationic lipid combined a T-cell activating vaccine.

In another embodiment, a method of treating cancer, the method comprises the step of treating the subject with a cationic lipid combined with a protein or peptide tumor antigen, and in combination with an adjuvant.

In another embodiment, a method of treating cancer, the method comprises the step of treating the subject with a cationic lipid combined with any tumor antigen including a DNA or RNA-based antigen.

In another embodiment, a method of treating cancer, the method comprises the step of treating the subject with a cationic lipid combined with a tumor antigen in combination with an adjuvant and/or any agent that combats tumor immune suppression via reduction of MDSC, Tregs or blocking of check point inhibitors.

In another embodiment, a method of treating cancer, the method comprises the step of treating the subject with a cationic lipid-based vaccine combined with a DNA or RNA-based tumor antigen in combination with an adjuvant and/or any agent that combats tumor immune suppression.

In yet another embodiment, a method of augmenting an anti-tumor immune response in a mammal is provided. The method comprises the step of treating the mammal with a cationic lipid-based vaccine with one or more cationic lipids together with growth factors in some cases such as GM-CSF and cytokines.

In the various embodiments, the composition comprises one or more lipids with at least one cationic lipid and at least one antigen.

### Antigens

In one embodiment, a cationic lipid is administered with autologous antigens such as antigens derived from the patient's own tumor. In another embodiment, the cationic lipid is administered in combination with non-autologous antigen(s) such as synthetic peptides, recombinant proteins, RNA or DNA. In each case the objective is to alter the tumor's microenvironment by reducing the Treg population within and around the tumor and by generating a T-cell immune response, which is specific to the antigen(s). The antigen can be any tumor-associated antigen known to one skilled in the art.

A “tumor-associated antigen,” as used herein is a molecule or compound (e.g., a protein, peptide, polypeptide, lipoprotein, lipopeptide, glycoprotein, glycopeptides, lipid, glycolipid, carbohydrate, RNA, and/or DNA) associated with a tumor or cancer cell and which is capable of provoking an immune response (humoral and/or cellular) when expressed on the surface of an antigen presenting cell in the context of an MHC molecule. Tumor-associated antigens include self-antigens, as well as other antigens that may not be specifically associated with a cancer, but nonetheless enhance an immune response to and/or reduce the growth of a tumor or cancer cell when administered to an animal. More specific embodiments are provided herein.

A “microbial antigen,” as used herein, is an antigen of a microorganism and includes, but is not limited to, infectious virus, infectious bacteria, infectious parasites and infectious fungi. Microbial antigens may be intact microorganisms, and natural isolates, fragments, or derivatives thereof, synthetic compounds which are identical to or similar to naturally-occurring microbial antigens and, preferably, induce an immune response specific for the corresponding microorganism (from which the naturally-occurring microbial antigen originated). In a preferred embodiment, a compound is similar to a naturally-occurring microorganism antigen if it induces an immune response (humoral and/or cellular) similar to a naturally-occurring microorganism antigen. Compounds or antigens that are similar to a naturally-occurring microorganism antigen are well known to those of ordinary skill in the art such as, for example, a protein, peptide, polypeptide, lipoprotein, lipopeptide, glycoprotein, glycopeptides, lipid, glycolipid, carbohydrate, RNA, and/or DNA. Another non-limiting example of a compound that is similar to a naturally-occurring microorganism antigen is a peptide mimic of a polysaccharide antigen. More specific embodiments are provided herein.

The term “antigen” is further intended to encompass peptide or protein analogs of known or wild-type antigens such as those described in this specification. The analogs may be more soluble or more stable than wild type antigen and may also contain mutations or modifications rendering the antigen more immunologically active. Antigen can be modified in any manner, such as adding lipid or sugar moieties, mutating peptide or protein amino acid sequences, mutating the DNA or RNA sequence, or any other modification known to one skilled in the art. Antigens can be modified using standard methods known by one skilled in the art.

Also useful in the compositions and methods of the present disclosure are peptides or proteins which have amino acid sequences homologous with a desired antigen's amino acid sequence, where the homologous antigen induces an immune response to the respective tumor, microorganism or infected cell.

In one embodiment, the antigen administered with the cationic lipid comprises an antigen associated with a tumor or cancer, i.e., a tumor-associated antigen, to make a vaccine to prevent or treat a tumor. As such, in one embodiment, the methods and compositions of the present disclosure further comprise at least one epitope of at least one tumor-associated antigen. In another embodiment, the methods and compositions of the present disclosure further comprise a plurality of epitopes from one or more tumor-associated antigens. The tumor-associated antigens used with the cationic lipids and methods of the present invention can be inherently immunogenic, or non-immunogenic, or slightly immunogenic. As demonstrated herein, even tumor-associated self-antigens may be advantageously employed in the subject immunotherapies for therapeutic effect, since the subject compositions are capable of breaking immune tolerance against such antigens by lowering the Treg population within the tumor. Exemplary antigens include, but are not limited to, synthetic, recombinant, foreign, or homologous antigens, and antigenic materials may include but are not limited to proteins, peptides, polypeptides, lipoproteins, lipopeptides, lipids, glycolipids, carbohydrates, RNA and DNA. Examples of such therapies include, but are not limited to, the treatment or prevention of breast cancer, head and neck cancer, melanoma, cervical cancer, lung cancer, prostate cancer gut carcinoma, or any other cancer known in the art susceptible to immunotherapy. In such therapies it is also possible to combine the antigen with the cationic lipid without encapsulation.

Tumor-associated antigens suitable for use in connection with the novel methods and compositions disclosed herein include both naturally occurring and modified molecules which may be indicative of single tumor type, shared among several types of tumors, and/or exclusively expressed or overexpressed in tumor cells in comparison with normal cells. In addition to proteins, glycoproteins, lipoproteins, peptides, and lipopeptides, tumor-specific patterns of expression of carbohydrates, gangliosides, glycolipids, and mucins have also been documented. Exemplary tumor-associated antigens for use in cancer vaccines include protein products of oncogenes, tumor suppressor genes, and other genes with mutations or rearrangements unique to tumor cells, reactivated embryonic gene products, oncofetal antigens, tissue-specific (but not tumor-specific) differentiation antigens, growth factor receptors, cell surface carbohydrate residues, foreign viral proteins, and a number of other self-proteins.

Specific examples of tumor-associated antigens include, but are not limited to, e.g., mutated or modified antigens such as the protein products of the Ras p21 protooncogenes, tumor suppressor p53 and HER-2/neu and BCR-abl oncogenes, as well as CDK4, MUM1, Caspase 8, and Beta catenin; overexpressed antigens such as galectin 4, galectin 9, carbonic anhydrase, Aldolase A, PRAME, Her2/neu, ErbB-2 and KSA, oncofetal antigens such as alpha fetoprotein (AFP), human chorionic gonadotropin (hCG); self-antigens such as carcinoembryonic antigen (CEA) and melanocyte differentiation antigens such as Mart 1/Melan A, gp100, gp75, Tyrosinase, TRP1 and TRP2; prostate associated antigens such as PSA, PAP, PSMA, PSM-P1 and PSM-P2; reactivated embryonic gene products such as MAGE 1, MAGE 3, MAGE 4, GAGE 1, GAGE 2, BAGE, RAGE, and other cancer testis antigens such as NY-ESO1, SSX2 and SCP1; mucins such as Muc-1 and Muc-2; gangliosides such as GM2, GD2 and GD3, neutral glycolipids and glycoproteins such as Lewis (y) and globo-H; and glycoproteins such as Tn, Thompson-Freidenreich antigen (TF) and sTn. Also included as tumor-associated antigens herein are whole cell and tumor cell lysates as well as immunogenic portions thereof, as well as immunoglobulin idiotypes expressed on monoclonal proliferations of B lymphocytes for use against B cell lymphomas.

Tumor-associated antigens and their respective tumor cell targets include, e.g., cytokeratins, particularly cytokeratin 8, 18 and 19, as antigens for carcinoma. Epithelial membrane antigen (EMA), human embryonic antigen (HEA-125), human milk fat globules, MBr1, MBr8, Ber-EP4, 17-1A, C26 and T16 are also known carcinoma antigens. Desmin and muscle-specific actin are antigens of myogenic sarcomas. Placental alkaline phosphatase, beta-human chorionic gonadotropin, and alpha-fetoprotein are antigens of trophoblastic and germ cell tumors. Prostate specific antigen is an antigen of prostatic carcinomas, carcinoembryonic antigen of colon adenocarcinomas. HMB-45 is an antigen of melanomas. In cervical cancer, useful antigens could be encoded by human papilloma virus. Chromagranin-A and synaptophysin are antigens of neuroendocrine and neuroectodermal tumors. Of particular interest are aggressive tumors that form solid tumor masses having necrotic areas. The lysis of such necrotic cells is a rich source of antigens for antigen-presenting cells, and thus the subject therapy may find advantageous use in conjunction with conventional chemotherapy and/or radiation therapy.

Tumor-associated antigens can be prepared by methods well known in the art. For example, these antigens can be prepared from cancer cells either by preparing crude extracts of cancer cells (e.g., as described in Cohen et al., Cancer Res., 54:1055 (1994)), by partially purifying the antigens, by recombinant technology, or by de novo synthesis of known antigens. The antigen may also be in the form of a nucleic acid encoding an antigenic peptide in a form suitable for expression in a subject and presentation to the immune system of the immunized subject. Further, the antigen may be a complete antigen, or it may be a fragment of a complete antigen comprising at least one epitope.

Antigens derived from pathogens known to predispose to certain cancers may also be advantageously included in the cancer vaccines of the present invention. It is estimated that close to 16% of the worldwide incidence of cancer can be attributed to infectious pathogens; and a number of common malignancies are characterized by the expression of specific viral gene products. Thus, the inclusion of one or more antigens from pathogens implicated in causing cancer may help broaden the host immune response and enhance the prophylactic or therapeutic effect of the cancer vaccine. Pathogens of particular interest for use in the cancer vaccines provided herein include the, hepatitis B virus (hepatocellular carcinoma), hepatitis C virus (heptomas), Epstein Barr virus (EBV) (Burkitt lymphoma, nasopharynx cancer, PTLD in immunosuppressed individuals), HTLVL (adult T cell leukemia), oncogenic human papilloma viruses types 16, 18, 33, 45 (adult cervical cancer), and the bacterium Helicobacter pylori (B cell gastric lymphoma). Other medically relevant microorganisms that may serve as antigens in mammals and more particularly humans are described extensively in the literature, e.g., C. G. A Thomas, Medical Microbiology, Bailliere Tindall, Great Britain 1983, the entire contents of which is hereby incorporated by reference.

In another embodiment, the antigen comprises an antigen derived from or associated with a pathogen, i.e., a microbial antigen. As such, in one embodiment, compositions of the present disclosure further comprise at least one epitope of at least one microbial antigen. Pathogens that may be targeted by the subject immunotherapies include, but are not limited to, viruses, bacteria, parasites and fungi. In another embodiment, the compositions of the present disclosure further comprise a plurality of epitopes from one or more microbial antigens.

The microbial antigens useful in the cationic lipid immunotherapies and methods disclosed herein may be inherently immunogenic, or non-immunogenic, or slightly immunogenic. Exemplary antigens include, but are not limited to, synthetic, recombinant, foreign, or homologous antigens, and antigenic materials may include but are not limited to proteins, peptides, polypeptides, lipoproteins, lipopeptides, lipids, glycolipids, carbohydrates, RNA, and DNA.

Exemplary viral pathogens include, but are not limited to, viruses that infect mammals, and more particularly humans. Examples of virus include, but are not limited to: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Also, gram negative and gram positive bacteria may be targeted by the subject compositions and methods in vertebrate animals. Such gram positive bacteria include, but are not limited to Pasteurella species, Staphylococci species, and Streptococcus species. Gram negative bacteria include, but are not limited to, Escherichia coli, Pseudomonas species, and Salmonella species. Specific examples of infectious bacteria include but are not limited to: Helicobacter pyloris, Borella burgdorferi, Legionella pneumophiliaii, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus infuenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatumii, Streptobacillus moniliformis, Treponema pallidium, Treponema pertenue, Leptospira, Rickettsia, and Actinomyces israelli.

Polypeptides of bacterial pathogens which may find use as sources of microbial antigens in the subject compositions include but are not limited to an iron-regulated outer membrane protein, (“IROMP”), an outer membrane protein (“OMP”), and an A-protein of Aeromonis salmonicida which causes furunculosis, p57 protein of Renibacterium salmoninarum which causes bacterial kidney disease (“BKD”), major surface associated antigen (“msa”), a surface expressed cytotoxin (“mpr”), a surface expressed hemolysin (“ish”), and a flagellar antigen of Yersiniosis; an extracellular protein (“ECP”), an iron-regulated outer membrane protein (“IROMP”), and a structural protein of Pasteurellosis; an OMP and a flagellar protein of Vibrosis anguillarum and V. ordalii; a flagellar protein, an OMP protein, aroA, and purA of Edwardsiellosis ictaluri and E. tarda; and surface antigen of Ichthyophthirius; and a structural and regulatory protein of Cytophaga columnari; and a structural and regulatory protein of Rickettsia. Such antigens can be isolated or prepared recombinantly or by any other means known in the art.

Examples of pathogens further include, but are not limited to, fungi that infect mammals, and more particularly humans. Examples of fungi include, but are not limited to: Cryptococcus neoformansi, Histoplasma capsulatum, Cocci dioides immitis, Blastomyces dermatitidis, Chlamydia trachomatis, Candida albicans. Examples of infectious parasites include Plasmodium such as Plasmodium falciparum, Plasmodium malariae, Plasmodium ovale, and Plasmodium vivax. Other infectious organisms (i.e. protists) include Toxoplasma gondii. Polypeptides of a parasitic pathogen include but are not limited to the surface antigens of Ichthyophthirius.

Other medically relevant microorganisms that serve as antigens in mammals and more particularly humans are described extensively in the literature, e.g., see C. G. A Thomas, Medical Microbiology, Bailliere Tindall, Great Britain 1983, the entire contents of which is hereby incorporated by reference. In addition to the treatment of infectious human diseases and human pathogens, the compositions and methods of the present invention are useful for treating infections of nonhuman mammals. Many vaccines for the treatment of non-human mammals are disclosed in Bennett, K. Compendium of Veterinary Products, 3rd ed. North American Compendiums, Inc., 1995; see also WO 02/069369, the disclosure of which is expressly incorporated by reference herein.

Exemplary non-human pathogens include, but are not limited to, mouse mammary tumor virus (“MMTV”), Rous sarcoma virus (“RSV”), avian leukemia virus (“ALV”), avian myeloblastosis virus (“AMV”), murine leukemia virus (“MLV”), feline leukemia virus (“FeLV”), murine sarcoma virus (“MSV”), gibbon ape leukemia virus (“GALV”), spleen necrosis virus (“SNV”), reticuloendotheliosis virus (“RSV”), simian sarcoma virus (“SSV”), Mason-Pfizer monkey virus (“MPMV”), simian retrovirus type 1 (“SRV-1”), lentiviruses such as HIV-1, HIV-2, SIV, Visna virus, feline immunodeficiency virus (“FIV”), and equine infectious anemia virus (“EIAV”), T-cell leukemia viruses such as HTLV-1, HTLV-II, simian T-cell leukemia virus (“STLV”), and bovine leukemia virus (“BLV”), and foamy viruses such as human foamy virus (“HFV”), simian foamy virus (“SFV”) and bovine foamy virus (“BFV”).

In some embodiments, “treatment,” “treat,” and “treating,” as used herein with reference to infectious pathogens, refer to a prophylactic treatment which increases the resistance of a subject to infection with a pathogen or decreases the likelihood that the subject will become infected with the pathogen; and/or treatment after the subject has become infected in order to fight the infection, e.g., reduce or eliminate the infection or prevent it from becoming worse.

Microbial antigens can be prepared by methods well known in the art. For example, these antigens can be prepared directly from viral and bacterial cells either by preparing crude extracts, by partially purifying the antigens, or alternatively by recombinant technology or by de novo synthesis of known antigens. The antigen may also be in the form of a nucleic acid encoding an antigenic peptide in a form suitable for expression in a subject and presentation to the immune system of the immunized subject. Further, the antigen may be a complete antigen, or it may be a fragment of a complete antigen comprising at least one epitope.

In order to improve incorporation of the antigen into the cationic lipid vesicles and also to improve delivery to the cells of the immune system, the antigen may be modified to increase its hydrophobicity or the negative charge on the antigen. Hydrophobicity of an antigen may be increased such as, for example, by conjugating to a lipid chain or hydrophobic amino acids in order to improve it's the antigen's solubility in the hydrophobic acyl chains of the cationic lipid, while maintaining the antigenic properties of the molecule. The modified antigen can be a lipoprotein, a lipopeptide, a protein or peptide modified with an amino acid sequence having increased hydrophobicity, and combinations thereof. The modified antigen may have a linker conjugated between the lipid and the antigen such as, for example, an N-terminal .alpha. or .epsilon.-palmitoyl lysine may be connected to antigen via a dipeptide serine-serine linker. Further, the antigen may be manipulated to increase its negative charge by altering the formulation buffer in which the antigen is encapsulated into the cationic lipid complexes or by covalently attaching anionic moieties such as, for example, anionic amino acids to the antigen.

In some embodiments described herein, the cationic lipid may be in the form of nanoparticle assemblies. As used herein, the term “nanoparticle” refers to a particle having a size measured on the nanometer scale. As used herein, the “nanoparticle” refers to a particle having a structure with a size of less than about 10,000 nanometers. In some embodiments, the nanoparticle is a liposome.

As used herein, the term “cationic lipid” refers to any of a number of lipid species which carry a net positive charge at physiological pH or have a protonatable group and are positively charged at pH lower than the pKa. Exemplary cationic lipids according to the present disclosure may include, but are not limited to: 3-.beta.[.sup.4N-(.sup.1N, .sup.8-diguanidino spermidine)-carbamoyl]cholesterol (BGSC); 3-.beta. [N,N-diguanidinoethyl-aminoethane)-carbamoyl]cholesterol (BGTC); N,N. sup. 1N. sup .2N. sup.3Tetra-methyltetrapalmityl spermine (cellfectin); N-t-butyl-N′-tetradecyl-3-tetradecyl-aminopropion-amidine (CLONfectin); dimethyldioctadecyl ammonium bromide (DDAB); 1,2-dimyristyloxypropyl-3-dimethyl-hydroxy ethyl ammonium bromide (DMRIE); 2,3-dioleoyloxy-N-[2(sperminecarboxamido)ethyl]-N,N-dimethyl-1-p-ropanaminium trifluorocetate) (DOSPA); 1,3-dioleoyloxy-2-(6-carboxyspermyl)-propyl amide (DOSPER); 4-(2,3-bis-palmitoyloxy-propyl)-1-methyl-1H-imidazole (DPIM) N,N,N′,N′-tetramethyl-N,N′-bis(2-hydroxyethyl)-2,3-dioleoyloxy-1,4-butane-diammonium iodide) (Tfx-50); N-1-(2,3-dioleoyloxy) propyl-N,N,N-trimethyl ammonium chloride (DOTMA) or other N-(N,N-1-dialkoxy)-alkyl-N,N,N-trisubstituted ammonium surfactants; 1,2dioleoyl-3-(4′-trimethylammonio) butanol-sn-glycerol (DOBT) or cholesteryl (4′trimethylammonia) butanoate (ChOTB) where the trimethylammonium group is connected via a butanol spacer arm to either the double chain (for DOTB) or cholesteryl group (for ChOTB); DORI (DL-1,2-dioleoyl-3-dimethylaminopropyl-.beta.-hydroxyethylammonium) or DORIE (DL-1,2-O-dioleoyl-3-dimethylaminopropyl-.beta.-hydroxyethylammoniu-m) (DORIE) or analogs thereof as disclosed in WO 93/03709; 1,2-dioleoyl-3-succinyl-sn-glycerol choline ester (DOSC); cholesteryl hemisuccinate ester (ChOSC); lipopolyamines such as dioctadecylamidoglycylspermine (DOGS) and dipalmitoyl phosphatidylethanolamylspermine (DPPES), cholesteryl-3.beta.-carboxyl-amido-ethylenetrimethylammonium iodide, 1-dimethylamino-3-trimethylammonio-DL-2-propyl-cholesteryl carboxylate iodide, cholesteryl-3-O-carboxyamidoethyleneamine, cholesteryl-3-.beta.-oxysuccinamido-ethylenetrimethylammonium iodide, 1-dimethylamino-3 -trimethylammonio-DL-2-propyl-cholesteryl-3-.beta.-oxysu-ccinate iodide, 2-(2-trimethylammonio)-ethylmethylamino ethyl-cholesteryl-3-.beta.-oxysuccinate iodide, 3-.beta. -N-(N′,N′-dimethylaminoethane) carbamoyl cholesterol (DC-chol), and 3-.beta.-N-(polyethyleneimine)-carbamoylcholesterol; O,O′-dimyristyl-N-lysyl aspartate (DMKE); O,O′-dimyristyl-N-lysyl-glutamate (DMKD); 1,2-dimyristyloxypropyl-3-dimethyl-hydroxy ethyl ammonium bromide (DMRIE); 1,2-dilauroyl-sn-glycero-3-ethylphosphocholine (DLEPC); 1,2-dimyristoyl-sn-glycero-3-ethylphosphocholine (DMEPC); 1,2-dioleoyl-sn-glycero-3-ethylphosphocholine (DOEPC); 1,2-dipalmitoyl-sn-glycero-3-ethylphosphocholine (DPEPC); 1,2-di stearoyl-sn-glycero-3-ethylphosphocholine (DSEPC); 1,2-dioleoyl-3 -trimethylammonium propane (DOTAP); dioleoyl dimethylaminopropane (DODAP); 1,2-palmitoyl trimethylammonium propane (DPTAP); 1,2-distearoyl-3-trimethylammonium propane (DSTAP), 1,2-myristoyl-3-trimethylammonium propane (DMTAP); and sodium dodecyl sulfate (SDS). Furthermore, structural variants and derivatives of the any of the described cationic lipids are also contemplated.

In some embodiments, the cationic lipid is selected from the group consisting of DOTAP, DOTMA, DOEPC, and combinations thereof In other embodiments, the cationic lipid is DOTAP. In yet other embodiments, the cationic lipid is DOTMA. In other embodiments, the cationic lipid is DOEPC. In some embodiments, the cationic lipid is purified.

In some embodiments, the cationic lipid is an enantiomer of a cationic lipid. The term “enantiomer” refers to a stereoisomer of a cationic lipid which is a non-superimposable mirror image of its counterpart stereoisomer, for example R and S enantiomers. In various examples, the enantiomer is R-DOTAP or S-DOTAP. In one example, the enantiomer is R-DOTAP. In another example, the enantiomer is S-DOTAP. In some embodiments, the enantiomer is purified. In various examples, the enantiomer is R-DOTMA or S-DOTMA. In one example, the enantiomer is R-DOTMA. In another example, the enantiomer is S-DOTMA. In some embodiments, the enantiomer is purified. In various examples, the enantiomer is R-DOEPC or S-DOEPC. In one example, the enantiomer is R-DOEPC. In another example, the enantiomer is S-DOEPC. In some embodiments, the enantiomer is purified.

### Terms

It is to be noted that the term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively.

As used herein, the term “about” means a variability of 10% from the reference given, unless otherwise specified.

As used herein, the terms “subject” and “patient” are used interchangeably and include a mammal, e.g., a human, mouse, rat, guinea pig, dog, cat, horse, cow, pig, or non-human primate, such as a monkey, chimpanzee, baboon or gorilla.

As used herein, the terms “disease”, “disorder” and “condition” are used interchangeably, to indicate an abnormal state in a subject.

As used herein, the term “tumor microenvironment” means the physiological, biological, cellular environment within and around which the tumor exists. This includes, but is not limited to, the surrounding blood vessels, and immune cells, and extracellular matrix.

Unless defined otherwise in this specification, technical and scientific terms used herein have the same meaning as commonly understood by one of ordinary skill in the art and by reference to published texts, which provide one skilled in the art with a general guide to many of the terms used in the present application.

The compositions of the disclosure comprise an amount of a composition comprising one or more cationic lipids, optionally combined with one or more antigens, wherein such composition is effective for generating an immunogenic response in a subject. Specifically, the dosage of the composition to achieve a therapeutic effect will depend on factors such as the formulation, pharmacological potency of the composition, age, weight and sex of the patient, condition being treated, severity of the patient's symptoms, route of delivery, and response pattern of the patient. It is also contemplated that the treatment and dosage of the compositions may be administered in unit dosage form and that one skilled in the art would adjust the unit dosage form accordingly to reflect the relative level of activity. The decision as to the particular dosage to be employed (and the number of times to be administered per day) is within the discretion of the ordinarily-skilled physician and may be varied by titration of the dosage to the particular circumstances to produce the therapeutic effect. Further, one of skill in the art would be able to calculate any changes in effective amounts of the compositions due to changes in the composition components or dilutions. In one embodiment, the compositions may be diluted 2-fold. In another embodiment, the compositions may be diluted 4-fold. In a further embodiment, the compositions may be diluted 8-fold.

The effective amount of the compositions disclosed herein may, therefore, be about 1 mg to about 1000 mg per dose based on a 70 kg mammalian, for example human, subject. In another embodiment, the therapeutically effective amount is about 2 mg to about 250 mg per dose. In a further embodiment, the therapeutically effective amount is about 5 mg to about 100 mg. In yet a further embodiment, the therapeutically effective amount is about 25 mg to 50 mg, about 20 mg, about 15 mg, about 10 mg, about 5 mg, about 1 mg, about 0.1 mg, about 0.01 mg, about 0.001 mg.

The effective amounts (if administered therapeutically) may be provided on regular schedule, i.e., on a daily, weekly, monthly, or yearly basis or on an irregular schedule with varying administration days, weeks, months, etc. Alternatively, the therapeutically effective amount to be administered may vary. In one embodiment, the therapeutically effective amount for the first dose is higher than the therapeutically effective amount for one or more of the subsequent doses. In another embodiment, the therapeutically effective amount for the first dose is lower than the therapeutically effective amount for one or more of the subsequent doses. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every 2 weeks, about every 3 weeks, about every month, about every 2 months, about every 3 months and about every 6 months. The number and frequency of dosages corresponding to a completed course of therapy will be determined according to the judgment of a health-care practitioner.

The compositions may be administered by any route, taking into consideration the specific condition for which it has been selected. The compositions may be delivered orally, by injection, inhalation (including orally, intranasally and intratracheally), ocularly, transdermally (via simple passive diffusion formulations or via facilitated delivery using, for example, iontophoresis, microporation with microneedles, radio-frequency ablation or the like), intravascularly, cutaneously, subcutaneously, intramuscularly, sublingually, intracranially, epidurally, rectally, intravesically, and vaginally, among others.

The compositions may be formulated neat or with one or more pharmaceutical carriers and/or excipients for administration. The amount of the pharmaceutical carrier(s) is determined by the solubility and chemical nature of the peptides, chosen route of administration and standard pharmacological practice. The pharmaceutical carrier(s) may be solid or liquid and may incorporate both solid and liquid carriers/matrices. A variety of suitable liquid carriers is known and may be readily selected by one of skill in the art. Such carriers may include, e.g., dimethylsulfoxide (DMSO), saline, buffered saline, cyclodextrin, hydroxypropylcyclodextrin (HPβCD), n-dodecyl-β-D-maltoside (DDM) and mixtures thereof. Similarly, a variety of solid (rigid or flexible) carriers and excipients are known to those of skill in the art.

Although the compositions may be administered alone, they may also be administered in the presence of one or more pharmaceutical carriers that are physiologically compatible. The carriers may be in dry or liquid form and must be pharmaceutically acceptable. Liquid pharmaceutical compositions may be sterile solutions or suspensions. When liquid carriers are utilized, they may be sterile liquids. Liquid carriers may be utilized in preparing solutions, suspensions, emulsions, syrups and elixirs. In one embodiment, the compositions may be dissolved a liquid carrier. In another embodiment, the compositions may be suspended in a liquid carrier. One of skill in the art of formulations would be able to select a suitable liquid carrier, depending on the route of administration. The compositions may alternatively be formulated in a solid carrier. In one embodiment, the composition may be compacted into a unit dose form, i.e., tablet or caplet. In another embodiment, the composition may be added to unit dose form, i.e., a capsule. In a further embodiment, the composition may be formulated for administration as a powder. The solid carrier may perform a variety of functions, i.e., may perform the functions of two or more of the excipients described below. For example, a solid carrier may also act as a flavoring agent, lubricant, solubilizer, suspending agent, filler, glidant, compression aid, binder, disintegrant, or encapsulating material. In one embodiment, a solid carrier acts as a lubricant, solubilizer, suspending agent, binder, disintegrant, or encapsulating material. The composition may also be sub-divided to contain appropriate quantities of the compositions. For example, the unit dosage can be packaged compositions, e.g., packeted powders, vials, ampoules, prefilled syringes or sachets containing liquids.

In an embodiment, the compositions may be administered by a modified-release delivery device. “Modified-release” as used herein refers to delivery of the disclosed compositions which is controlled, for example over a period of at least about 8 hours (e.g., extended delivery) to at least about 12 hours (e.g., sustained delivery). Such devices may also permit immediate release (e.g., therapeutic levels achieved in under about 1 hour, or in less than about 2 hours). Those of skill in the art know suitable modified-release delivery devices.

Also provided are kits comprising the compositions disclosed herein. The kit may further comprise packaging or a container with the compositions formulated for the delivery route. Suitably, the kit contains instructions on dosing and an insert regarding the compositions.

A number of packages or kits are known in the art for dispensing pharmaceutical compositions for periodic use. In one embodiment, the package has indicators for each period. In another embodiment, the package is a foil or blister package, labeled ampoule, vial or bottle.

The packaging means of a kit may itself be geared for administration, such as an inhaler, syringe, pipette, eye dropper, catheter, cytoscope, trocar, cannula, pressure ejection device, or other such apparatus, from which the formulation may be applied to an affected area of the body, such as the lungs, injected into a subject, delivered to bladder tissue or even applied to and mixed with the other components of the kit.

One or more components of these kits also may be provided in dried or lyophilized forms. When reagents or components are provided as a dried form, reconstitution generally is by the addition of a suitable solvent. It is envisioned that the solvent also may be provided in another package. The kits may include a means for containing the vials or other suitable packaging means in close confinement for commercial sale such as, e.g., injection or blow-molded plastic containers into which the vials are retained. Irrespective of the number or type of packages and as discussed above, the kits also may include, or be packaged with a separate instrument for assisting with the injection/administration or placement of the composition within the body of an animal. Such an instrument may be an inhaler, syringe, pipette, forceps, measuring spoon, eye dropper, catheter, cytoscope, trocar, cannula, pressure-delivery device or any such medically approved delivery means.

The term “treat”, “treating”, or any variation thereof is meant to include therapy utilized to remedy a health problem or condition in a patient or subject. In one embodiment, the health problem or condition may be eliminated permanently or for a short period of time. In another embodiment, the severity of the health problem or condition, or of one or more symptoms characteristic of the health problem or condition, may be lessened permanently, or for a short period of time. The effectiveness of a treatment of pain can be determined using any standard pain index, such as those described herein, or can be determined based on the patient's subjective pain. A patient is considered “treated” if there is a reported reduction in pain or a reduced reaction to stimuli that should cause pain.

This invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention.

### EXAMPLES

It should be noted that for the purposes of illustration all examples are performed utilizing a model protein ovalbumin which has been well studied and which is available with a dual fluorescence label. The use of the model protein provides an excellent illustration of how cationic lipids enhance antigen uptake processing and presentation. Also, the availability of TCR transgenic T cells specific for the class I and class II restricted OVA peptides enables a detailed study and confirmation of antigen presentation via both routes.

The outlined examples shed light on the fact that cationic lipids may be effective in facilitating interaction with dendritic cells and also facilitating antigen presentation via the MHC Class I pathway. However, this characteristic is not predictive of the lipid's ability to mature and activate dendritic cells or to prime strong antigen-specific CD8+ T-cells to infiltrate the tumor microenvironment, and therefore not predictive of the ability of a cationic lipid to significantly alter the tumor's microenvironment by reducing the Treg to CD8+ T-cell ratio. All in vitro studies reported in the examples were performed using the model protein ovalbumin as a representative antigen. To assess the effects of cationic lipids on antigen uptake and processing by antigen presenting cells, fluorescent OVA conjugates (DQ-OVA conjugate, and Alexa Fluor® 647 OVA conjugate) were used, which can be easily traced using flow cytometer. In addition, use of Ovalbumin protein as antigen facilitated confirmation of antigen presentation via MHCI and MHC II using Ovalbumin-specific T cell hybridoma cells and TCR transgenic mice (OT-1 and D011.10) bearing ovalbumin specific CD4 and CD8 T cell receptors. The results shown in this study are applicable in general to all protein and peptide antigens.

### Example 1

### Effect of Cationic Lipids on Antigen Processing by Dendritic Cells and Epithelial Cells

To determine the effects of cationic lipids on antigen uptake and processing by dendritic cells, a fluorescent ovalbumin protein called DQ-OVA was used. DQ-OVA is non-fluorescent when intact, but emits both red and green fluorescence when the protein is degraded. Dendritic cells were grown from mouse bone marrow by culturing for 8 days in GMCSF+IL-4 (hereafter referred to as BMDC). BMDC were incubated at 37° C. or 4° C. for 1 hour with DQ-OVA alone, or DQ-OVA mixed with different concentrations of the cationic lipid R-DOTAP. The cells were then washed, fixed, and stained with fluorescent antibodies to CD11c, a marker for dendritic cells. Cells were then analyzed on an LSRII flow cytometer in both red and green fluorescent channels.

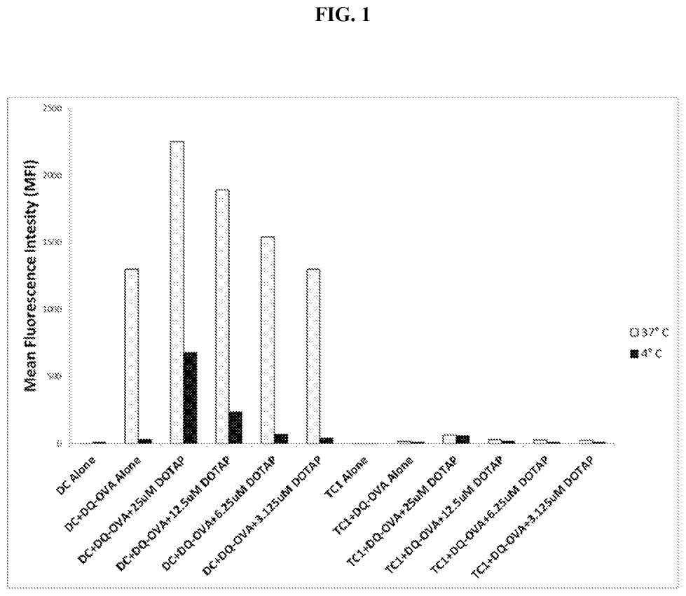

Results in FIG. 1 show that BMDC incubated with DQ-OVA in media alone showed enhanced fluorescence at 37° C. indicating uptake and processing. This represents the well-known mannose receptor mediated uptake of OVA by DC. This uptake and processing was inhibited at 4° C. confirming that active cytoskeletal rearrangements are required for this type of uptake. BMDC incubated with DQ-OVA in the presence of DOTAP showed a significant increase of fluorescence indicating that DOTAP greatly enhances protein uptake and processing in DC. Significant uptake was even seen at 4° C. indicating that DOTAP can facilitate protein uptake in the absence of active cellular metabolism. The effect of DOTAP was concentration dependent with 50 uM showing the greatest effect.

To determine if the DOTAP-induced enhancement of protein uptake is cell dependent, we incubated a mouse epithelial cell line with DQ-OVA under identical conditions as the BMDC. Results in Figure lshow that this uptake and processing of OVA is only observed in DC and not in epithelial cells.

This data indicates that DOTAP can greatly enhance the uptake and processing of a whole protein into dendritic cells ex-vivo/in-vivo. Further, it indicates that this enhancement is selective for antigen presenting cells such as dendritic cells and not other, non-antigen presenting cell types.

### Example 2

### Comparison of Effect of Cationic Lipids on Antigen Processing and Endosomal Entry with Known Adjuvants

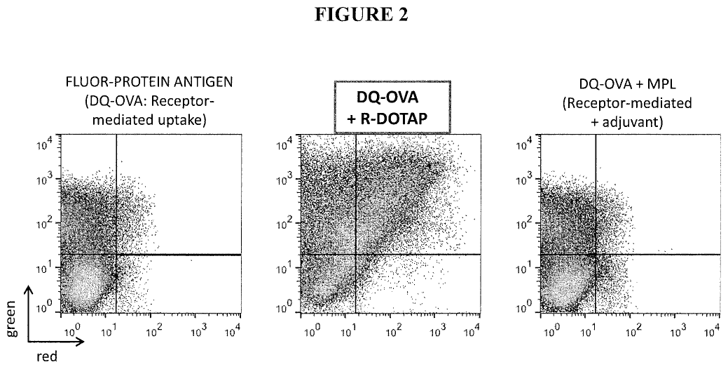

To determine whether non-cationic lipid adjuvants could mediate the same effect as DOTAP, BMDC were incubated with DQ-OVA in media alone or with DOTAP as described for FIG. 1. In addition, BMDC were incubated under identical conditions with the potent lipid adjuvant lipopolysaccharide (MPL). As shown in FIG. 2, DQ-OVA was actively taken up and processed by DC in the absence of R-DOTAP, but uptake was greatly enhanced in the presence of R-DOTAP and manifested as a strong increase in red fluorescence. In contrast, no such enhancement was observed with MPL treatment.

Monophorphoryl lipid-A (MPL) is a lower toxicity derivative of LPS that is now an FDA approved adjuvant in several vaccines.

It should also be noted that when the study was performed with S-DOTAP no difference in ability to enhance DQ-OVA uptake and processing was observed between R-DOTAP and S-DOTAP. A similar effect to R-and S-DOTAP was noted with other cationic lipids including DOTMA, DDA and DOEPC.

These data suggest that the ability of R- and S-DOTAP to facilitate protein uptake into DC is not a general property of lipids or adjuvants, but rather a unique property of cationic lipids.

### Example 3

### Effect of Cationic Lipids on Antigen Processing and Cross-Presentation to MHC Class I Restricted T Cells

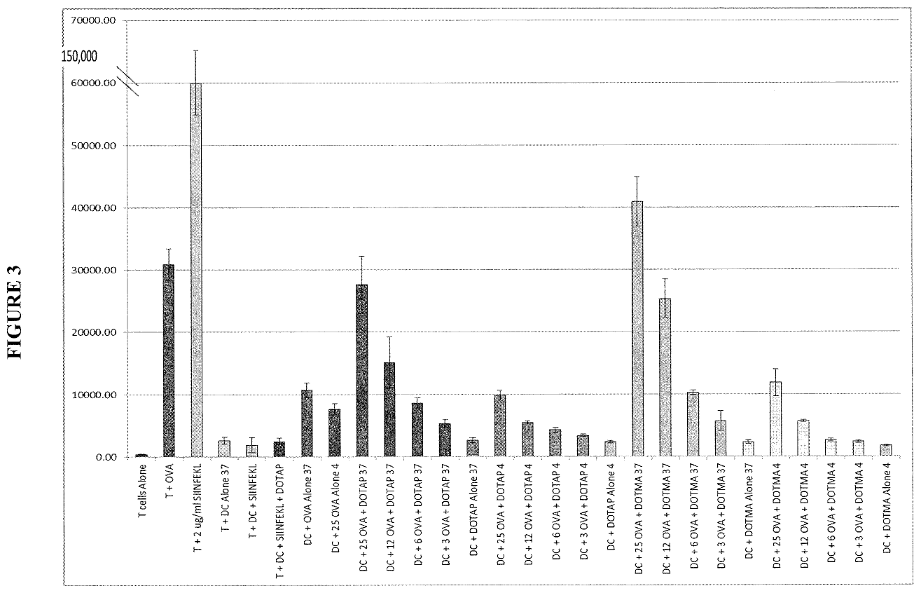

In order to verify that the cationic lipid facilitated uptake of antigen translates into enhanced antigen presentation on MHC class I (cross presentation), T cells from a TCR transgenic mouse (OT-1) were utilized in which all T cells are specific for an internal peptide of OVA. These T-cells will only proliferate if presented with DCs which have processed OVA and presented an OVA peptide on MHC class I molecules. Thus, this represents a stringent assay for cross-presentation. BMDC were incubated with different concentrations of the whole OVA protein in the presence or absence of two cationic lipids, either DOTAP or DOTMA for 1 hour at 37° C. The DCs were then washed, fixed and added to the OVA peptide specific T-cells. The results in FIG. 3 show that DC incubated with OVA in the presence of the cationic lipids DOTAP or DOTMA cross-presented antigen to the CD8+ T cells much more efficiently than DC incubated with OVA without cationic lipid. This response was dose dependent with respect to the OVA concentration, and was even apparent when DC were incubated with OVA at 4° C.

These results demonstrate that the enhanced uptake of antigen mediated by cationic lipids results in superior processing of antigen and entry of peptides into MHC class I pathway, an absolute prerequisite for effective priming and activation of tumor-targeting CD8+ T cells.

### Example 4

### Evaluation of Antigen-Specific In-Vivo CD8+ T-Cell Induction by R-DOTAP and S-DOTAP

C57 black mice were vaccinated with various formulations:

Group 1: KF18 HPV peptide

Group 2: KF18 HPV peptide+R-DOTAP liposomes

Group 3: KF18 HPV peptide+S-DOTAP liposomes

Group 4: KF18 peptide+MPL/Alum adjuvant

*For each of the groups above, the KF18 HPV peptide corresponds to Palmitoyl-KSS-GQAEPDRAHYNIVTF (SEQ ID NO: 2)

5 mice per group were injected with the various formulations. The mice were vaccinated on Day 0 and Day 7 and sacrificed on Day 14. The splenocytes were removed and ELISPOT studies performed. The splenocytes were stimulated with the peptide RAHYNIVTF (SEQ ID NO: 3), the HPV16 CD8+ T-cell epitope peptide recognized by the C57 mice.

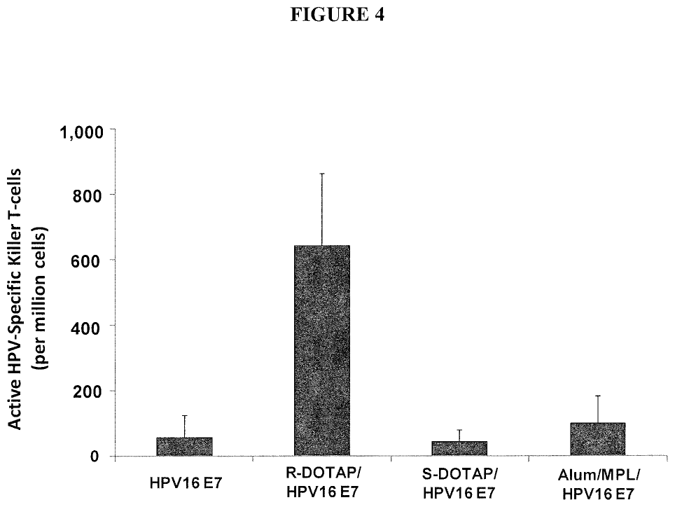

The studies demonstrate that R-DOTAP was effective in inducing strong HPV-specific CD8+ T-cell responses. However, S-DOTAP which demonstrated identical ability to promote antigen uptake, internalization and processing, as well as maturation of dendritic cells, did not result in an enhanced CD8+ T-cell response beyond what was seen with the peptide alone (FIG. 4). MPL was ineffective in promoting antigen uptake compared to both R-DOTAP and S-DOTAP, hence the significantly lower CD8+ T-cell response compared to R-DOTAP was expected.

The studies suggest that the reported ability of cationic lipids to facilitate antigen uptake and presentation does not necessarily lead to immune activation and induction of a strong antigen-specific T-cell response which is needed to effectively induce CD8+ T-cells which can infiltrate into the tumor's microenvironment to induce apoptosis and killing of the antigen-specific tumor cells.

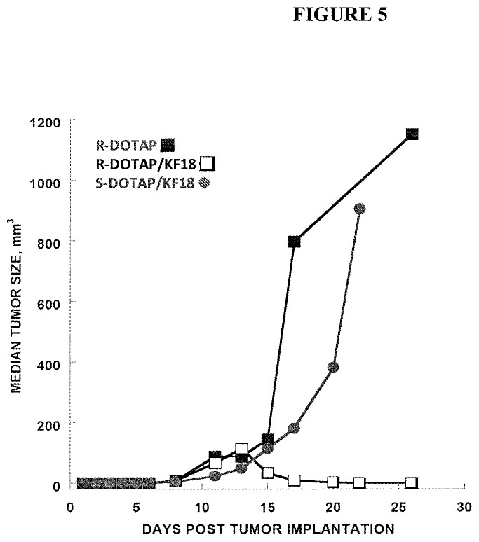

In tumor regression studies to compare the anti-tumor effects of R and S-DOTAP, 1×10E⁵ TC-1 tumor cells were injected into the flank of the mice on day 0. R-DOTAP only, R-DOTAP/HPV16 E7 (SEQ ID NO: 2) and S-DOTAP/HPV16 E7 (SEQ ID NO: 2) were administered on Day 6 after tumor implantation. FIG. 5 shows the results of the study with R-DOTAP/E7 showing potent anti-tumor effect and S-DOTAP/E7 showing a lack of anti-tumor efficacy.

An additional example of this effect is observed with the cationic lipid DDA. DDA has been demonstrated to facilitate antigen uptake and presentation similarly to R- and S-DOTAP. However, it has been reported that to induce strong antigen-specific T-cell responses DDA has to be used in combination with strong adjuvants (Brandt L. et al, ESAT-6 Subunit Vaccination against Mycobacterium tuberculosis, Infect Immun. 2000 Feb; 68(2): 791-795).

### Example 5

### Comparison of R-DOTAP, GM-CSF Adjuvant and Vaccines Based on R-DOTAP and GM-CSF on the Population of Regulatory T cells and Antigen-Specific CD8+ T-cells within the Tumor Microenvironment

Due to the observation of enhanced antigen uptake and presentation by R-DOTAP as well as the strong CD8+ T-cell induction in-vivo (FIG. 4), a head to head study was performed to compare the ability of R-DOTAP and GM-CSF based immunotherapies alone, and when combined with specific tumor antigens to alter the tumor microenvironment. The tumor microenvironment was evaluated for the presence of immune-suppressive regulatory T-cells (Treg), and for the presence of antigen-specific CD8+ T-cells. The resulting effects on the regression of established HPV-positive TC-1 tumors were also studied. GM-CSF is a powerful T-cell adjuvant that has been evaluated with tumor antigens as a cancer vaccine in human clinical trials.

C57 mice were divided into the following groups of 8 mice per group:

Group 1: R-DOTAP+KF18 (SEQ ID NO: 2)

Group 2: GM-CSF+KF18 (SEQ ID NO: 2)

Group 3: R-DOTAP

Group 4: GM-CSF

Group 5: KF18 (SEQ ID NO: 2)

Group 6: Naive mice (tumor bearing and untreated).

1×10E⁵ TC-1 tumor cells were injected into the flank of the mice on day 0. The various formulations were administered on Days 12 and 19 after tumor implantation.

### Tregs:

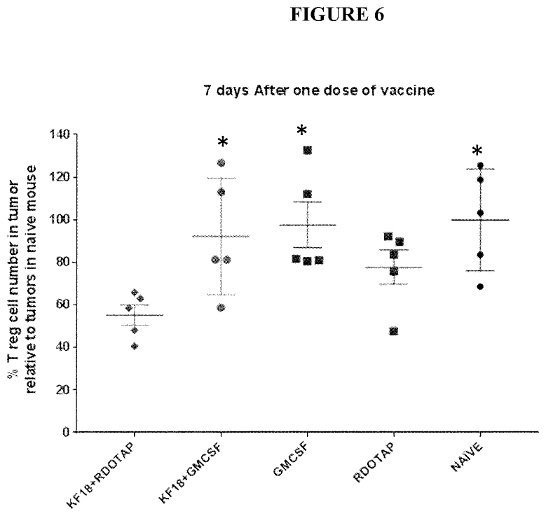

On Day 19 (1 week after vaccination) flow cytometry was used to study the impact of treatment on the immuno-suppressive tumor microenvironment, specifically the regulatory T cell population (CD45+ CD3+CD4+CD25+Foxp3+cells). The results are summarized in FIG. 6. The study demonstrates that a significant reduction in the Treg population within the tumors of about 20% with R-DOTAP only and about 40% with R-DOTAP+KF18 (SEQ ID NO: 2) is observed within 1 week of vaccination. A statistically significant reduction (P<0.01) in Tregs exist between R-DOTAP+KF18 (SEQ ID NO: 2) and all other groups except the R-DOTAP only group.

### CD8+ T-cells

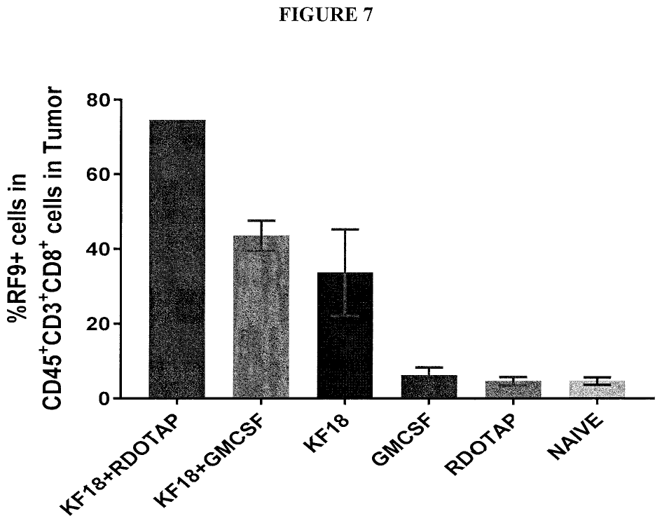

Antigen specific T cells infiltrating into the tumor were measured using RF9 specific dextramers specific for the CD8+ peptide epitope (SEQ ID NO: 3) code named RF9 for this study, and flow cytometry (FIG. 7). These CD8+ T cells were measured as a percentage of all immune cells (CD45+, CD3+and CD8+) present in the tumor. A significantly enhanced CD8+ T-cell count is observed within the tumor microenvironment with the R-DOTAP+KF18 group (Group 1) compared to all other groups including GM-CSF+KF18.

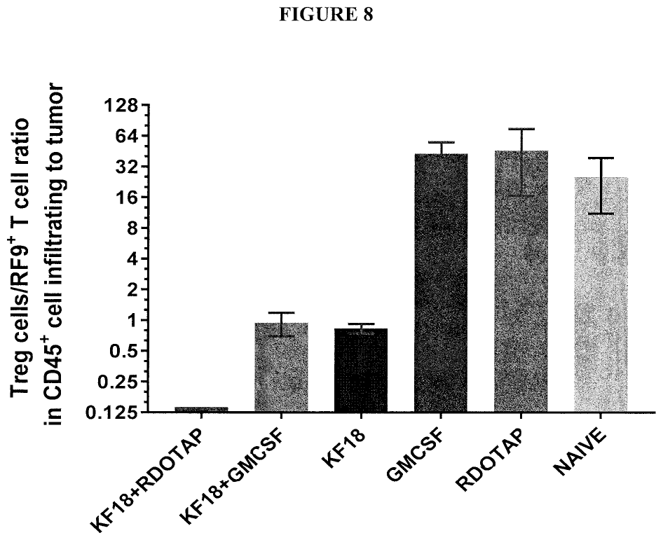

Of critical importance to the clinical efficacy of any immunotherapy is the ratio of immune suppressive cells to tumor targeting CD8+ T cells within the tumor microenvironment. A lower ratio of immune suppressor cells to CD8+ T cells promotes improved prognosis for anti-tumor benefit. This example shows a dramatically reduced Treg/CD8+ T-cell ratio of less than 0.13 for R-DOTAP +KF18 (SEQ ID NO:2) compared to a ratio of approximately 1 for GM-CSF+KF18 (SEQ ID NO:2) and for KF18 (SEQ ID NO:2) antigen only. The groups without tumor antigen exhibited a ratio of approximately 32 (FIG. 8). R-DOTAP promotes the preferential expansion of the right phenotype of effector T-cells in preference to Tregs. This leads to a significant modification of the tumor microenvironment leading to “a shift in power” in favor of the CD8+ T-cells the attackers” over the immuno-suppressive Tregs “defenders”, and thus highly effective immunotherapy.

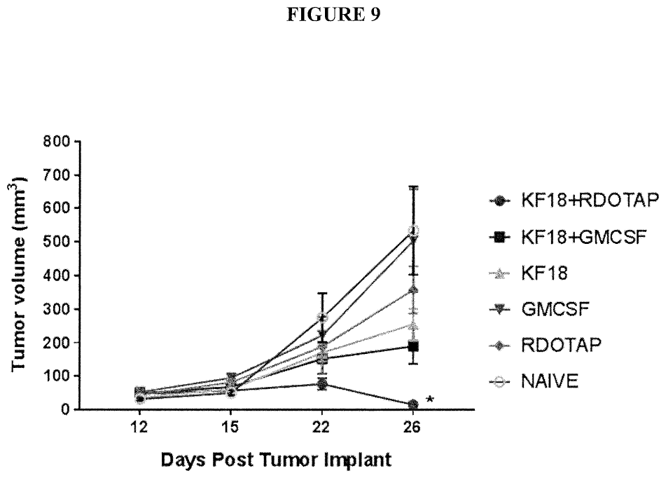

FIG. 9 shows that the animals treated with R-DOTAP+KF18 (SEQ ID NO:2) (Treg/CD8+ratio<0.13) all had complete elimination of their tumors by Day 26. GM-CSF+KF18 (SEQ ID NO:2) and KF18 antigen only groups (Treg/CD8+ ratio of approx. 1.0), both did not induce any tumor regression but rather inhibited tumor growth leading to a tumor volume of about 200 mm³ on Day 26. The third group of animals who were treated with either R-DOTAP alone or GM-CSF alone without antigen, or left untreated (Treg/CD8+ ratio>30) had tumor volumes of 300-700 mm³. The superior anti-tumor immune response correlates with the superior effect in altering the tumor's microenvironment with the reduced population of immune suppressive Treg cells increased population of HPV-specific CD8+ T-cells, and hence significantly reduced Treg to CD8+ T-cell ratio.

### Example 6

### Evaluation of R-DOTAP Vaccination on T And B-Cell Infiltration into the Lymph Nodes

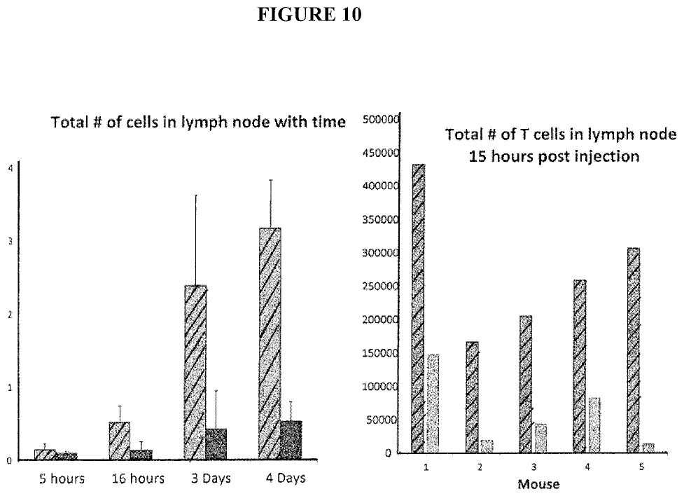

To better understand the ability of T-cell activating cationic lipids such as R-DOTAP to promote the induction of the effector CD8+ T-cells, which are also critical in altering the tumor's microenvironment, the impact of R-DOTAP vaccination on B and T-cells was studied. 12 mM R-DOTAP or sucrose as control were injected into the right and left foot pad respectively of mice and the influx of T-cells and total lymphocytes into the draining lymph nodes were quantified by flow cytometry. In this experiment, 15 hours after vaccination the popliteal lymph nodes were removed and analysis performed. FIG. 10 shows that R-DOTAP induced significant infiltration of T-cells into the lymph node. In a second experiment the analysis was performed at 5 hours, 16 hours, 3 days and 4 days and lymphocyte infiltration into the lymph nodes was seen to increase over the 4-day period (FIG. 10). Five mice were used per study. The cationic lipid-induced influx of T-cells into the draining lymph nodes facilitates the presentation of CD8+ T-cell epitope peptides via HMC Class I pathway to T-cells, hence facilitating effective priming of antigen-specific CD8+ T-cells.

### Example 7

### Investigating the Ability of R-DOTAP to Induce Chemoattractant Chemokines and Their Role in Lymphocyte Infiltration into the Lymph Nodes

The primary objective of the current experiment was to understand if T-cell infiltration induced by R-DOTAP is chemokine dependent. We utilized 5 mice to perform the study, and visualized the homing of CFSE labeled adoptively transferred cells. The study included a population of cells that had been treated in vitro with pertussis toxin to inactivate chemokine receptors. Pertussis toxin-treated and untreated cells were labeled with two different concentrations of CFSE so that they could be distinguished by flow cytometry. If the DOTAP enhanced homing is due to chemokines, the pertussis toxin population should not be present, or should be present only at greatly reduced levels in the DLN.

Spleen cells were prepared from a single B6 mouse and divided in half. Half of the cells were treated with Pertussis toxin 100 ng/ml for 1 hour at 37° C. and washed. The two cell populations were then labeled with CFSE at two different concentrations so they could be distinguished by flow cytometry, and mixed together. The mix (10e7 cells) was injected i.v. into the tail vein of 5 B6 mice. The mice were then anesthetized and injected in the footpad with either sucrose (right footpad) or R-DOTAP (left footpad, 50 ul, 600 nmoles).

After 16 h, the mice were sacrificed and the popliteal LN and spleens harvested. The total cells recovered from left and right nodes from each mouse were counted. The transferred CFSE labeled lymphocytes infiltrated the lymph node upon R-DOTAP vaccination. However, this did not occur with the pertussis treated cells, indicating that the cationic lipids induce the influx of lymphocytes into the lymph nodes and this phenomenon is most probably chemokine mediated.

Previous studies (see for example U.S. Pat. No. 8,877,206) suggested that cationic lipids induce chemokines CCL2, 3 and 4. However, these chemokines are not involved in lymph node homing. The study therefore suggests that the cationic lipids such as R-DOTAP specifically induce lymph node homing chemokines such as CCL21 or CXCL12.

Although the invention has been described with reference to the presently preferred embodiment, it should be understood that various modifications can be made without departing from the spirit of the invention. Accordingly, the invention is limited only by the following claims.

What is claimed:

1. A method for altering a tumor microenvironment comprising administering to a subject having a tumor, a composition comprising a cationic lipid.

2. The method of claim 1, further comprising autologous antigens, non-autologous antigens, or tumor-associated antigens.

3. The method of claim 1, wherein altering the tumor microenvironment comprises reducing the population of Tregs.

4. The method of claim 1, wherein altering the tumor microenvironment comprises both reducing the population of Tregs and increasing the population of CD8+ T-cells.

5. The method of claim 1, wherein altering the tumor microenvironment comprises reducing the Treg to CD8+ T-cell ratio.

6. The method of claim 1, wherein the composition further comprises an adjuvant or an agent that combats tumor immune suppression.

6. ethod of claim 6, further comprising a T-cell activating vaccine.

8. The method of claim 2, wherein the composition further comprises DNA-based antigens, RNA-based antigen, growth factors, GM-CSF, cytokines, synthetic peptides, recombinant proteins, or epitopes from one or more tumor-associated antigens.

9. The method of claim 1, wherein the cationic lipid is selected from the group consisting of DOTAP, R-DOTAP, S-DOTAP, DOTMA, R-DOTMA, S-DOTMA, DOEPC, R-DOEPC, and S-DOEPC.

10. The method of claim 3, wherein the cationic lipid consists of R-DOTAP and wherein the composition further comprises a tumor-associated antigen.

11. The method of claim 1, wherein altering the tumor microenvironment comprises improving antigen presentation to CD8+ T-cells via MEW class I, inducing chemoattractant chemokines to promote priming of T-cells, inducing proliferation of tumor-infiltrating T-cells, or reducing immune suppressive cell populations within the tumor microenvironment.

12. A method of improving cancer treatment comprising combining a cancer treatment regimen with a method for altering a tumor microenvironment,
wherein the method for altering a tumor microenvironment comprises administering to a subject having a tumor, a composition comprising a cationic lipid.

13. The method of claim 12, further comprising autologous antigens, non-autologous antigens, or tumor-associated antigens.

14. The method of claim 12, wherein altering the tumor microenvironment comprises reducing the population of Tregs.

15. The method of claim 12, wherein altering the tumor microenvironment comprises both reducing the population of Tregs and increasing the population of CD8+ T-cells.

16. The method of claim 12, wherein altering the tumor microenvironment comprises reducing the Treg to CD8+ T-cell ratio.

17. The method of claim 12, wherein the cationic lipid further comprises, DNA-based antigens, RNA-based antigen, growth factors, GM-CSF, cytokines, synthetic peptides, recombinant proteins, or epitopes from one or more tumor-associated antigens.

18. The method of claim 12, wherein the cationic lipid is selected from the group consisting of DOTAP, R-DOTAP, S-DOTAP, DOTMA, R-DOTMA, S-DOTMA, DOEPC, R-DOEPC, and S-DOEPC.

19. The method of claim 12, wherein altering the tumor microenvironment comprises improving antigen presentation to CD8+ T-cells via MEW class I, inducing chemoattractant chemokines to promote priming of T-cells, inducing proliferation of tumor-infiltrating T-cells, or reducing immune suppressive cell populations within the tumor microenvironment.

20. The method of claim 14, wherein the cationic lipid consists of R-DOTAP and wherein the composition further comprises a tumor-associated antigen.


SEQUENCE LISTING (3 sequences)

SEQ ID NO: 1 - protein, 8 aa, synthetic construct
  REGION 1..8: note = Synthetic
SIINFEKL

SEQ ID NO: 2 - protein, 18 aa, synthetic construct
  REGION 1..18: note = Synthetic
KSSGQAEPDRAHYNIVTF

SEQ ID NO: 3 - protein, 9 aa, synthetic construct
  REGION 1..9: note = Synthetic
RAHYNIVTF